(19) United States
(12) Patent Application Publication
(10) Pub. No.: US 2017/0176423 A1
(43) Pub. Date: Jun. 22, 2017

(54) MAGNETIC PROGRAMMABLE BEAD ENZYME-LINKED IMMUNOSORBENT ASSAY

(71) Applicant: ARIZONA BOARD OF REGENTS ON BEHALF OF ARIZONA STATE UNIVERSTIY, Scottsdale, AZ (US)

(72) Inventors: Karen ANDERSON, Scottsdale, AZ (US); Julia CHENG, Tempe, AZ (US); Joshua LABAER, Chandler, AZ (US)

(73) Assignee: Arizona Board of Regents on behalf of Arizona State University, Scottsdale, AZ (US)

(21) Appl. No.: 15/128,412

(22) PCT Filed: Mar. 18, 2015
(86) PCT No.: PCT/US2015/021188

§ 371 Date: Sep. 22, 2016

Related U.S. Application Data
(60) Provisional application No. 61/970,285, filed on Mar. 25, 2014.

(51) Int. Cl. G01N 33/543; C12N 15/62

(52) U.S. Cl. CPC G01N 33/5434; C12N 15/62

(57) ABSTRACT

Methods for making and using a programmable bead composition that in some embodiments includes a magnetic bead coupled to an antibody with DMTMM and an antigen coupled to the antibody, with the antigen having been expressed from a human cell lysate (FIG. 1). An ELISA method with the composition includes contacting a fluid sample from a patient with the magnetic programmable bead composition, the composition comprising a plurality of magnetic beads coupled to an antibody with MM and being bound to an antigen expressed from a human call lysate, and adding a detection antibody under conditions and for a time to measure the detection antibody.

### CROSS-REFERENCE TO RELATED APPLICATIONS

This application claims priority to U.S. Provisional Patent Application No. 61/970,285 filed on Mar. 25, 2014.

### STATEMENT OF GOVERNMENT RIGHTS

This invention was made with government support under U01 CA117374 awarded by the National Institute of Health. The government has certain rights in the invention.

### TECHNICAL FIELD

This invention relates to biomarker validation system methods and materials and more particularly to those involving a magnetic platform.

### BACKGROUND

Genomic advances over recent years have markedly improved our understanding of the molecular alterations that drive disease development. Technical innovations in proteomics have translated this knowledge into a rapid pace of discovery of potential biomarkers for the early detection and prognosis of cancer. Despite significant progress in targeted therapeutics that have reached clinical practice, the development of the vast majority of biomarkers for clinical use have been stalled after the discovery stage. This is due, in part, because the downstream assays required for validation of selected targets call for automation, stringent accuracy, and years of development. In addition, the high cost of these discovery platforms prohibits validation of biomarkers across hundreds and thousands of serum samples. As a result, few biomarkers have been approved by the FDA.

Of potential biomarkers, cancer-specific autoantibodies have been identified using multiple proteomic discovery platforms such as protein arrays, reverse-phase protein arrays, and phage display. Autoantibody biomarkers have been discovered in the sera of patients with breast cancer, colorectal cancer, prostate cancer, ovarian cancer, lung cancer, and many other cancer types. The altered protein structures derived from carcinogenesis (mutations, splice variation, overexpression) can result in a robust immune response-although the mechanisms, specificity, and functional consequences of that immune response remain poorly understood. While promising, these candidate biomarkers require validation using well-annotated, blinded, multicenter sample collections that focus on the target clinical applications, using methods that are easily reproducible in a mid-high throughput setting. Ideally, validation assays would also use reagents and equipment that are readily available and cost-effective for the research community to test these markers with their own samples.

### SUMMARY

This disclosure relates to methods for making and using a magnetic programmable bead composition that includes a magnetic bead coupled to an antibody with 4-(4,6-Dimethoxy-1,3,S-triazin-2-yl)-4-methylmorpholinium chloride (DMTMM) and an antigen coupled to the antibody, with the antigen having been expressed from a human cell lysate.

In one embodiment, an ELISA method with the composition includes contacting a fluid sample from a patient with the magnetic programmable bead composition, the composition comprising a plurality of magnetic beads coupled to an antibody with DMTMM and being bound to an antigen expressed from a human call lysate, and adding a detection antibody under conditions and for a time sufficient to measure the detection antibody.

In another embodiment, an in situ expression and capture of target-antigens method is disclosed. The method includes expressing protein in vitro from cDNA with human cell lysate, wherein the cDNA encodes and is expressed into a fusion protein in the lysate and coupled with an antibody that binds a portion of the fusion protein, and wherein the antibody is coupled to a substrate. For example, the substrate can be a bead or plate.

Other features and advantages of the invention will be apparent from the following detailed description and figures, and from the claims.

### DESCRIPTION OF DRAWINGS

FIG. 1. (A) Schematic of coupling anti-tag Ab to a 96-well plate. Individual cDNA's encoding antigens are expressed in vitro as fusion proteins using human cell lysate IVTT system. The proteins are captured in a well of the plate by the coupled antibodies. After addition of sera (or plasma), bound Ig is detected with labeled secondary Abs and luminescence is measured. (B) Schematic of the MagProBE for detection of serum Ig. Individual cDNA's encoding antigens are expressed in vitro as fusion proteins using human cell lysate IVTT system. The proteins are captured onto magnetic beads via anti-tag antibodies that are covalently coupled onto the beads. For multiplexed assays, protein-loaded beads are mixed. After addition of sera (or plasma), bound Ig is detected with PE-labeled secondary Abs and mean fluorescence index (MFI) is measured.

FIG. 2. Binding capacity, stability, and protein yield of the MagProBE. (A) Protein binding capacity of beads. Anti-GST was covalently attached to magnetic beads using either EDC or DMTMM coupling chemistry. Purified recombinant S100A7-GST or S100A7-HIS were added, and bound protein was detected with anti-GST-PE. (B) Protein expression using human Hela-cell lysate. HPV16gene products and GST were expressed in vitro using human cell lysate IVTT, and captured onto anti-GST coupled beads. Bound GST protein was detected with secondary antibody. Dotted line: Net MFI of anti-GST coupled beads with mock DNA.

FIG. 3. Multiplexed detection of HPV16-specific Abs in human sera. The 8 genes of HPV16 were expressed as 9 GST-fused gene products (E2 was expressed as NE2 and CE2 fragments), with GST control protein. Protein-loaded beads were mixed, and plasma from an HPV16-positive oropharyngeal cancer patient or healthy control plasma were added. Bound IgG was measured as net MFI. Error bars are present but not visible due to extremely low variability between duplicates.

FIG. 4. Specific detection of Abs to p53 or HPV16 in cancer patient sera. (A) Detection of HPV16 E6 Abs in oropharyngeal cancer patient sera (N=32), and healthy controls (N=15), in plate-based ELISA and beady array ELISA. Y-axis: the ratio of specific antigen MFI to control GST protein (or RLU ratio to control protein as labled). (B) Detection of p53 Abs in serous ovarian cancer patient sera (N=30) and healthy controls (N=30).

### DETAILED DESCRIPTION

Autoantibody validation requires assessing the levels of selected autoantibodies amongst thousands of unrelated antibodies in sera. A common and accessible ELISA assay in widespread research and clinical use are multiplexed bead-based arrays, which display covalently attached target antigens as sandwich ELISAs for cytokine detection. Bead arrays have variable reproducibility, limiting the application of these platforms. Recently, magnetic bead arrays have been developed to improve automation.

These assays require target antigen production, purification, and coupling to beads for serologic screening. The variable stability, purity, epitopes, and yield of purified proteins limit rapid protein preparation for biomarker validation. Our previous studies have demonstrated that protein bead arrays for multiplexed serologic assays can be rapidly generated using optimized vectors to express proteins with rabbit reticulocyte lysate. Because this assay is based on cDNA rather than purified protein, the speed and flexibility permits development of serologic assays within days while using publicly-available reagents. However, the bead array ELISA system has been limited by cost ($92/antigen/serum), reproducibility (inter-assay CV's of 7-21%), and lack of the automation that is required for high-throughput serologic testing.

Here, we have adapted the in-situ ELISA to a magnetic bead array platform, termed MagProBE (Magnetic Programmable Bead ELISA), using human cellular lysates for protein production. Using defined sera with known antibodies for the cancer-specific antigens p53 and HPV16 E6, we demonstrate the sensitivity, specificity, and improved reproducibility of these assays to detect cancer-specific biomarkers.

The invention will be further described in the following examples, which do not limit the scope of the invention described in the claims.

### EXAMPLES

Human papillomavirus (HPV) type 16-positive oropharyngeal cancer (OPC) patient sera and controls used in these analyses were collected from the Dana-Farber Cancer Institute (DFCI) and Mount Sinai School of Medicine (MSSM). Ovarian cancer patient sera used in these analyses were collected from the Brigham and Women's Hospital and the DFCI with support from the National Cancer Institute Early Detection Research Network. Samples were obtained at the time of presentation prior to surgery. After written informed consent, samples were collected using standardized serum and plasma collection protocols under institutional review board approval and stored at −80° C. until use.

Sequence verified, full-length cDNA expression plasmids (pANT7_cGST) containing Gateway-compatible donor systems were obtained from the DNASU Plasmid Repository at The Biodesign Institute in Arizona State University, and are publicly available online (http:ljdnasu.asu.edu/DNASU/). DNA was purified using a Nucleobond Maxiprep Kit (Ciontech Laboratories, Inc., Mountain View, Calif.). The GST-expressing plasmid pANT7_GST was used as the control.

Full length HPV16 genes E1, E4, ES, E6, E7, L1, and L2 and the tumor antigen pS3 were obtained by PCR and inserted into the pANT7_cGST vector as described in the art. The HPV16 E2 gene was expressed as N- and C-terminal fragments for optimal protein expression as described in the art.

Anti-GST antisera (GE Healthcare, Piscataway, N.J.) were dialyzed into PBS to remove sodium azide and coupled to Magplex magnetic beads (BioRad, Hercules, Calif.) using standard EDC (1-ethyl-3-(3-dimethylaminopropyl) carbodiimide) covalent coupling (Thermo Scientific, Rockford, Ill.) according to known protocols at a ratio of 5 μg antibody (Ab) per 1 million beads, using a DynaMag-2 (Life Technologies, Grand Island, N.Y.) to pellet the beads. An alternative covalent coupling method used 4-(4,6-Dimethoxy-1,3,S-triazin-2-yl)-4-methylmorpholinium chloride (DMTMM) (Sigma-Aldrich, St. Louis, Mo.) as described in the art.

The carboxyl groups were activated by incubating beads with 50 μl of 50 mg/mL DMTMM dissolved in 50 mM monobasic sodium phosphate, pH 5.0, for 20 minutes at room temperature (RT) with gentle mixing by sonication at 10-minute intervals. After washing, beads were resuspended with 1 mL of Ab solution at a concentration of 3.2 3.2 μg Ab per 1million beads in coupling buffer. The solution was incubated at room temperature while rotating, followed by three washes with PBS-TBN (PBS, 0.1% BSA, 0.02% Tween-20, 0.05% sodium azide, pH 7.4), and stored in PBS-TBN at 2-8 C with protection from light. Final bead counts were measured by a TC10 automated cell counter (BioRad, Hercules, Calif.). This method is applicable to coupling Ab up to 50 million beads with an average bead recovery rate of 70.6%. Ab coupling was confirmed with R-phycoerythrin (PE)-conjugated donkey anti-goat immunoglobulin G (IgG) Ab (Jackson lmmunoResearch Laboratories, Inc., West Grove, Pa.).

Full-length recombinant S100A7-GST protein was generated in Escherchia coli using pGEX-4XT and purified using glutathione beads as described. S100A7-GST protein was titered from 60.28 nM to 58.94 pM and incubated with 2000 anti-GST coupled beads produced by either the EDC- or DMTMM-coupling protocol, per reaction. Murine anti-GST mAb (Cell Signaling Technology, Inc., Danvers, Mass.) and PE-labeled goat anti-mouse IgG Ab (Jackson ImmunoResearch Laboratories) were used to detect bound GST protein.

Recombinant S100A7-HIS tagged protein was generated in E. coli using the pQE30 plasmid and purified with a nickel NTA column (Qiagen, Valencia, Calif.) as described and was used as the control. GST-tagged proteins were individually expressed from 500 ng DNA using in vitro transcription/translation (IVTT) with human Hela cell lysate (Thermo Scientific, Waltham, Mass.) diluted at 40% using PBS in microfuge tubes. All other components were added at manufacturer's recommendations with an additional 3 ml of RNaseOUT (Life Technologies, Grand Island, N.Y.). DNA was incubated with the human cell lysate IVTT system and its components at 30° C. for 90 minutes. Anti-GST Ab-coupled magnetic beads (4000-5000 per sera, for duplicates (2000/well)) were blocked in PBS-1% BSA, followed by incubation at RT with expressed protein for 2 hours on a rotator with protection from light. Protein capture reactions were scaled up for multiple assays. After protein capture, beads were resuspended at 200 beads/μl with PBS-1% BSA.

For multiplexed assays, beads of different color regions with different loaded proteins were mixed and added to 96-well plates (Greiner Bio One, Monroe, NC) at 10 μl antigen/well. Beads were washed with PBS-1% BSA with an automated microplate washer (BioTek, Inc., Winooski, Vt.) using the Biotek MAGX2 program. Beads and diluted sera were blocked separately for land 2 hours, respectively, at RT rotating or shaking with a custom blocking buffer (active blocking reagent) (Heteroblock™ (Omega Biologicals, Bozeman, Mont.) diluted to 2 μg/ml into steelhead salmon serum in PBS, e.g., SeaBlock™ (Thermo Scientific, Rockford, Ill.). Proceeding blocking, sera and beads were incubated together on the 96-well plate on a shaker in 4·c for 16-18 hours. R-Phycoerythrin-labeled goat anti-human IgG Ab (Jackson ImmunoResearch Laboratories) was added as the detection Ab at a concentration of 6 μg/ml, and net median fluorescent intensity (MFI) was measured.

Patient sera from HPV-positive OPC cases and ovarian cancer cases were used to confirm the specificity and sensitivity of the MagProBE. HPV-positive patient and control sera were tested for IgG to express and capture HPV16 E6, and ovarian cancer patient and control sera were tested for IgG to expressed and captured p53. GST-only controls were used to confirm protein specificity.

MagProBE net MFI measurements were performed on a Magpix using Luminex xPONENT software (EMD Millipore, Billerica, Mass.), 50 counts per analysis. All assays were performed in duplicate, and values are plotted as mean values. Intra-assay variability was determined using fluorescent measurements of HPV16 EG IgG antibody detection ofthe same sera repeated in duplicates on 4 plates. These signals were averaged and the coefficient of variation (CV, (SD/mean)×100%) was calculated. Inter-assay variability was determined in a similar fashion on 4 consecutive days, and signals were averaged and used to calculate the CV. Cut-off values of positive sera were defined as an MFI ratio>(the average+3 Standard Deviations (SD)) of the control sera.

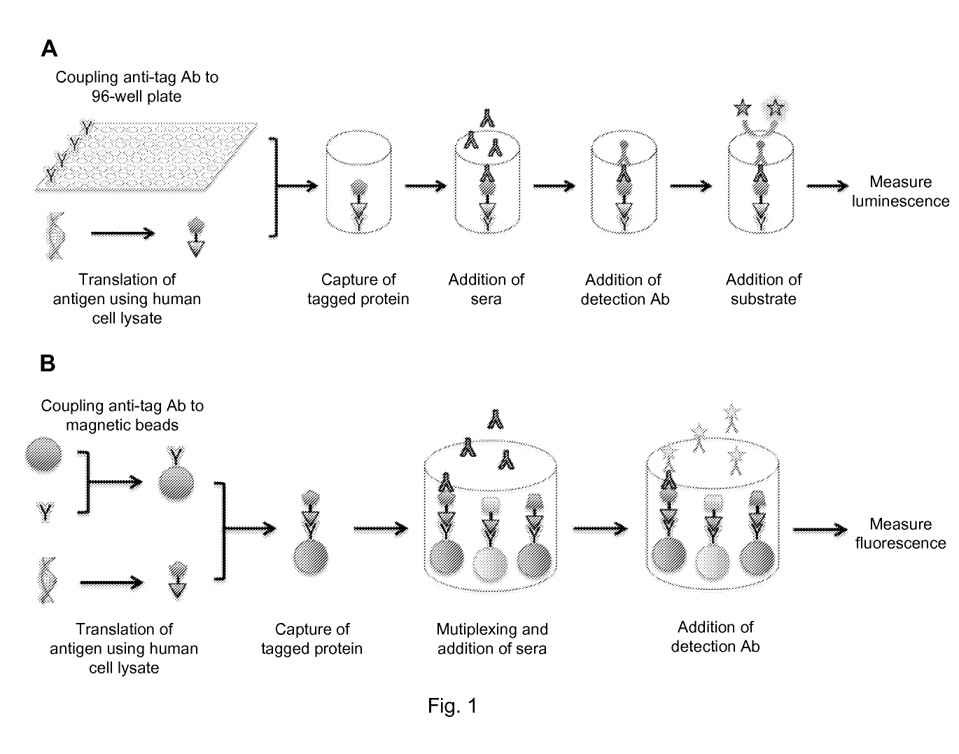

Magnetic bead arrays contain activated surface carboxyl groups for covalent attachment of ligands. They are internally labeled with fluorophores for multiplexed detection, and contain a 2-4% concentration of iron for compatibility with automated magnetic manipulation. As illustrated in FIG. 1, anti-tag Abs (anti-GST is used here) are covalently coupled to the bead arrays, and confirmed using secondary Abs specific for the capture Ab (data not shown). Separately, plasmids encoding target antigens are expressed using IVTT, and subsequently captured onto individual anti-GST coupled magnetic beads. Protein content is confirmed using anti-GST detection Abs. These protein-loaded beads are then pooled for multiplexed protein display. After addition of sera or plasma, bound Ig is detected with secondary Abs.

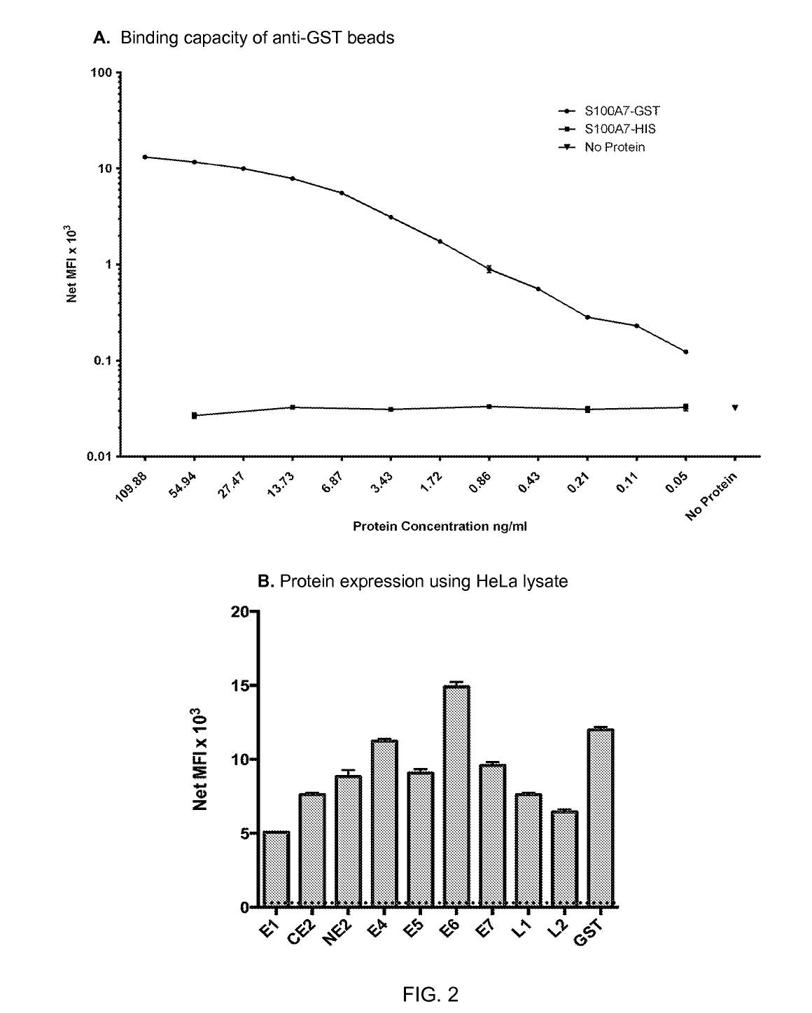

EDC-based coupling is the standard method for covalent attachment of proteins to bead arrays. We compared the relative efficiency and stability of anti-GST-coupled beads using EDC and DMTMM. Both EDC- and DMTMM-coupling methods yielded comparable coupling efficiency/mg Ig, as measured by anti-Ig secondary Abs (data not shown). Since covalent attachment of Abs to bead surfaces can alter their ligand binding capacity, we compared the efficiency of GST-protein capture between DMTMM-and EDC-based coupling. Recombinant, bacterially-expressed and purified GST-tagged protein (S100A7-GST) and control protein (S100A7-HIS) were added to anti-GST beads and detected using GST-specific secondary reagents. S100A7 is a known breast cancer tumor antigen, and is readily expressed and purified either as GST-tagged or HIS-tagged protein. DMTMM-coupled and EDC-coupled beads demonstrate similar binding characteristics over a 1000-fold range of protein concentration (FIG. 2A). Beads coupled with both methods were able to bind to a minimum of 60 nM of recombinant-GST protein per 2000 beads without significant decrease in Net MFI. Further increases in protein titration have shown the magnetic beads possess a binding capacity of 480 μM of recombinant-GST protein per 2000 beads at the highest Net MFI (data not shown). DMTMM-coupled beads were used for all further assays. The antibody-coupled beads are stable with an average decrease in signal intensity of 18% for the 10 analytes over two months (FIG. 2B).

Our previous bead array ELISAs relied on rabbit reticulocyte lysate IVTT. We have expressed over 10,000 proteins on protein arrays with >90% success but there are several limitations with this system. First, we have observed both batch-to-batch variation in protein expression (presumably due to differences in animal sources) and increased background in serologic assays, possibly due to xeno-specific Abs targeting glycosylated epitopes on the reticulocytes. Recently, a fully human-based IVTT system based on human Hela cell lysate has been developed. Hela lysate yields a 10-fold increase of in vitro protein expression, and can be diluted to 40% without significant loss of protein expression. The Hela cell lysate-driven protein expression and bead capture of 9 tumor antigens derived from the HPV16 proteome (E1, NE2, CE2, E4, ES, EG, E7, L1, and L2), as well as GST-expressing control vector is shown in FIG. 2C. The dotted line represents the background signal of mock expression (no DNA).

Non-magnetic SeroMap bead arrays have been optimized for detection of antibodies in sera. Despite this, in previous Ab studies we noted significant background issues when using human sera (data not shown). We had developed a custom blocking solution to further minimize nonspecific binding of IgG to the bead arrays, which included xenogeneic sera. In our initial studies using magnetic bead arrays, we observed twice the non-specific binding of human IgG to the arrays (data not shown). We compared the effects of commercially-available blocking solutions (HeteroBlock, SeaBlock, PVP/PVA (Sigma-Aidrich)/CBS-K (Chemicon, Millipore)) on signal-to-background ratios using a selected human serum containing both specific Abs to HPV16 EG and high background signal. Of the tested buffers, a combination of HeteroBlock at 2 μg/ml in SeaBlock added to both sera and beads resulted in two-fold increase in MFI ratio of detected IgG specific for E6 compared with GST control protein (data not shown).

One major impetus for switching to a magnetic platform was to improve the automation and the reproducibility of the assays. Our prior serologic assays for EBV-, p53- and HPV-derived proteins had intra-assay CVs of 0.2-22% and inter-assay CVs of 7-21%, limiting potential clinical application. To determine the CVs of the MagProBE assay, replicates for E6 serology were performed with 8 replicates on 4 consecutive days. MFI ratios (E6-GST: GST control) among bead arrays processed on the same day showed excellent reproducibility with intra-assay CVs of 1-8%. MFI ratios between bead arrays processed on 4 consecutive days demonstrated inter-assay CVs of 3 -7%.

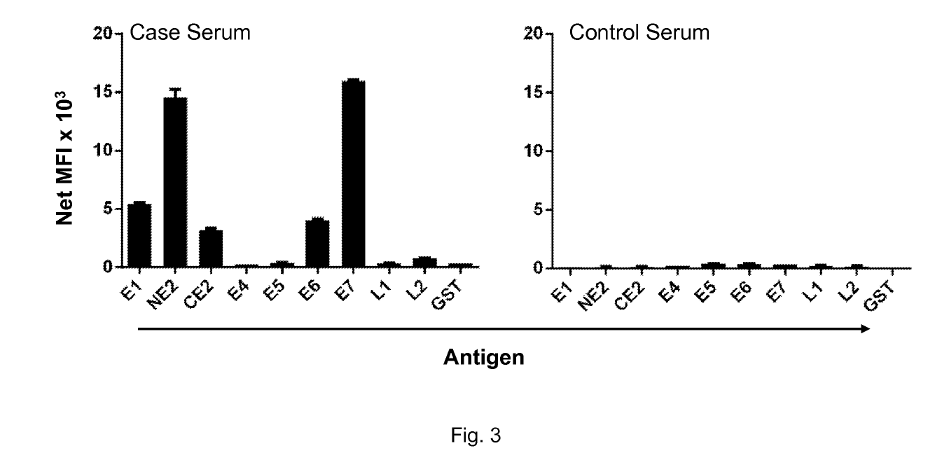

Alteration of structure-dependent epitopes during protein translation is a major theoretical concern regarding protein expression using xenogeneic systems. To determine if known immunogenic epitopes that were detected using rabbit reticulocyte lysate are retained by human Hela cell lysate expression, the proteome of HPV16 was expressed as 9 separate proteins (E2 is fragmented into N-and C-terminal parts for optimized protein expression) with GST control protein. These antigen-loaded beads were multiplexed and tested with plasma from a patient with HPV16-positive OPC and known specific Abs to HPV16 E1, NE2, CE2, E6, and E7, and with plasma from a healthy control. In FIG. 3a, Abs were specifically detected to all of the known immunogenic HPV16-derived proteins, demonstrating both retention of HPV-specific epitopes using human Hela cell lysate, and effective multiplexing of the MagProBE. Using serial dilutions of serum from a HPV16-positive patient with known Abs to HPV16E7, the optimal serum dilution for detection of Abs in serum was determined to be between 1:80-1:320 (FIG. 3b). More concentrated sera interfered with detection in this assay, which is consistent with the “hook effect” observation as seen in other immunoassays. All sera in this study's assays were diluted 1:80.

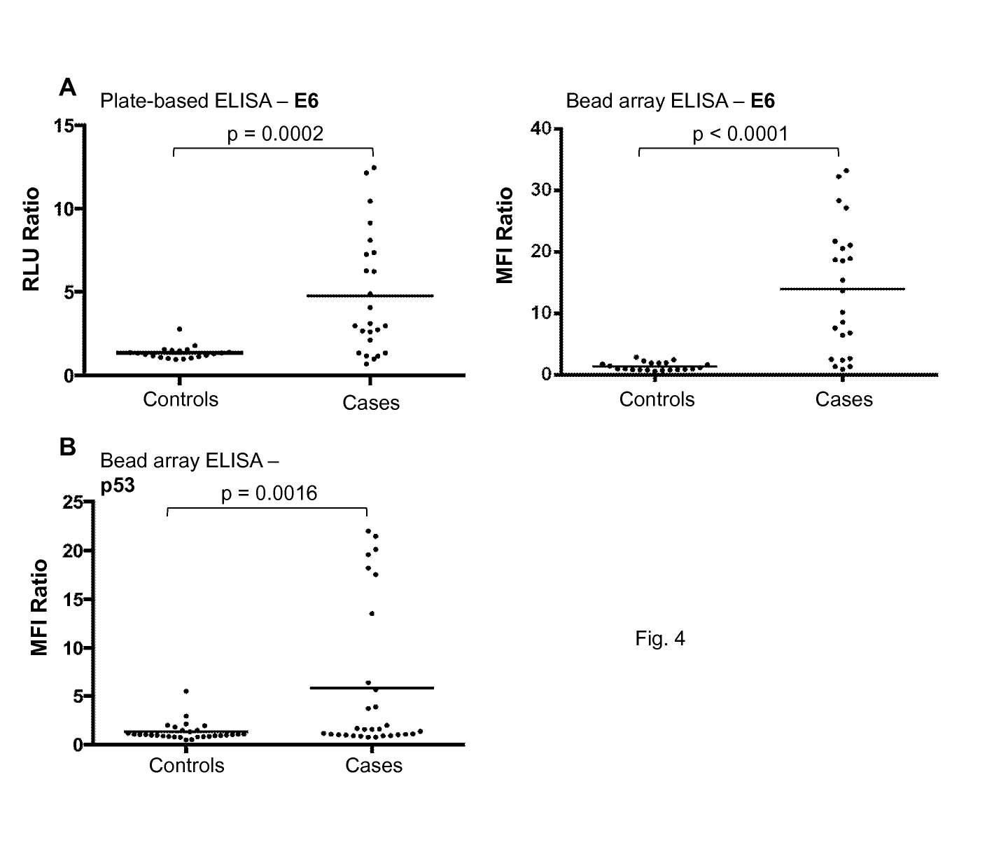

We and others have previously identified serum antibodies to two cancer proteins, HPV16 E6 and p53, with strong potential for clinical use for the early detection of HPV16-positive OPC and serous ovarian cancer, respectively. To confirm the specificity and sensitivity of the magnetic bead array ELISA for these known cancer biomarkers using validated, published sera, and sera from HPV-positive OPC patients (N=32) and healthy control sera (N=15) were measured for IgG to HPV16 E6 compared to GST control protein (FIG. 4A). E6 Abs were specifically detected in the OPC cancer patient sera (p<0.0001, mean MFI ratio 3.30, range 1.03-7.66) compared to healthy control sera (mean MFI ratio 1.14, ranges 0.83-1.50). Using cutoff values of the mean+2 SO of the control sera, E6 Abs were detected in 59.4% of cases at 100% specificity. Similarly, sera from serous ovarian cancer patients (N=30) and healthy gender-matched control sera (N=30) were measured for IgG to p53 proteins compared to GST control protein (FIG. 4B). p53-specific antibodies were detected in the ovarian cancer patient sera (p=0.0199, mean MFI ratio 5.28, range 0.37-24.3), compared with healthy controls (mean MFI ratio 1.34, range 0.30-9.33). p53 Abs were detected in 33.3% of ovarian cancer sera at 93.3% specificity. These results are consistent with prior published studies of these biomarkers.

We have developed and optimized a sensitive and specific programmable ELISA using magnetic bead arrays for the multiplexed serologic detection of antibody biomarkers to target antigens, termed MagProBE. Other alternatives for a commercially available multiplexed immunoassay platform exist, such as the MULTI-ARRAY platform (Meso Scale Discovery) multiplexed sandwich ELISA that uses electrochemiluminescent (ECL)-based detection to improve sensitivity.

However, the unique features of the MagProBE system include an optimized vector for mammalian-based protein expression, in situ expression and capture of target antigens using human cell lysates, and magnetic-based automation. The use of human cell lysates was associated with approximately a 10-fold increase in protein expression, decreased batch-to-batch variability, and decreased background. These modifications have significantly decreased inter-assay variability and cost, and we have confirmed specific detection of antibodies to the cancer-specific biomarkers p53 and HPV16 E6 in patient sera.

Using cDNA rather than purified protein, antibodies to potential tumor antigens can now be measured in hundreds of sera using a 2-day assay, with a final cost down from $90/antigen/day to $3/antigen/sera, utilizing materials that are publicly available. The MagProBE is a promising tool for research-based validation of any novel tumor antigen for which cDNA is available. The method of protein production for clinical serologic assays may be critical for the display of intact antigenic epitopes for antibody binding. Current technologies of bacterial and baculoviral expression systems, and likely human celllysates, involve limited post-translational modifications. Alterations in glycosylation are common features of human cancers, so antibodies to those epitopes may be highly specific for epithelial cancers, in particular. Preliminary studies of glycan arrays have identified antibodies to numerous glycan features in ovarian cancer and pancreatic cancer). Future development of methods for in vitro alteration of glycosylation after human lysate expression would be required to identify those epitopes on this platform.

Here, we confirmed the selective detection of antibodies in sera to two specific tumor antigens, HPV16 E6, and p53. Despite effective HPV vaccines, HPV infections are drastically increasing with approximately 20 million Americans currently infected and 6.2 million new infections per year. HPV has been identified as an etiological cause of a subset of OPC, with approximately 83,000 estimated cases per year worldwide. HPV16 is the dominant subtype, accounting for 85-90% of HPV-positive OPC. Unlike cervical disease, there are no current screening methods for the detection for OPC, and there is a need for validated diagnostic biomarkers for early diagnosis and intervention. Specific antibodies to HPV16 E6 have been detected in cervical cancer patients up to 5 years before diagnosis, suggesting serology may be useful as a diagnostic biomarker.

Previously, using in vitro translated and captured antigens, we had identified antibodies in sera to multiple early gene products from the HPV16 proteome. Here, using reference sera from that study, we confirmed that multiple antibodies to HPV16 early gene products are detected in patient sera. In addition, we confirmed that sera from 60% of patients with HPV16-positive OPC presented antibodies to HPV16 E6 at 100% specificity.

The other potential cancer biomarker that we confirmed in this assay are autoantibodies to p53, which is mutated in up to 50% of cancers, including ovarian, breast, lung, colon, and pancreatic cancers. A subset of patients with mutant p53 tumors (15-20%) have antibodies to wild-type p53 protein in their sera, which is highly specific. We had identified p53-specific antibodies in a subset of patients with serous ovarian cancer, and here confirmed that expression in human lysates was effective for p53 protein display and serologic detection of p53 autoantibodies.

In summary, the MagProBE is a flexible method for the validation of serologic detection of target antigens. While these studies have focused on using the displayed proteins for the detection of antibodies in human sera, the methods of protein display on a bead-array platform is amenable to other measurements of protein-protein and protein-DNA interactions.

Thus, it is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A magnetic programmable bead composition, comprising a magnetic bead coupled to an antibody with 4-(4,6-Dimethoxy-1,3,S-triazin-2-yl)-4-methylmorpholinium chloride.

2. The composition of claim 1, further comprising an antigen coupled to said antibody, with said antigen having been expressed from a human cell lysate.

3. An ELISA method, comprising:
contacting a fluid sample from a patient with a magnetic programmable bead composition, the composition comprising a plurality of magnetic beads, each of said bead being coupled to an antibody with 4-(4,6-Dimethoxy-1,3,S-triazin-2-yl)-4-methylmorpholinium chloride and an antigen expressed from a human call lysate; and
adding a detection antibody under conditions and for a time to measure an amount of said detection antibody.

4. An in situ expression and capture of target-antigens method, the method comprising expressing protein in vitro from cDNA with human cell lysate, wherein said cDNA encodes and is expressed into a fusion protein in said lysate and coupled with an antibody that binds a portion of said fusion protein, and wherein said antibody is coupled to a substrate.

5. The method of claim 5, wherein said substrate comprises a bead or plate.
